(19) United States
(12) Patent Application Publication
(10) Pub. No.: US 2011/0077610 A1
(43) Pub. Date: Mar. 31, 2011

(54) MALE INCONTINENCE ABSORBENT POUCH

(76) Inventors: NANCY KIKUMOTO, Pearl City, HI (US); Kyle Kikumoto, Pearl City, HI (US)

(21) Appl. No.: 12/894,133

(22) Filed: Sep. 29, 2010

Related U.S. Application Data
(60) Provisional application No. 61/246,552, filed on Sep. 29, 2009.

(51) Int. Cl. A61F 13/64

(52) U.S. Cl. 604/385.03

(57) ABSTRACT

An absorbent pouch used for collecting urine for those males that are unable to get to a toilet is provided. The absorbent pouch has at least two optional elastic bands and a pouch for collecting the urine. The absorbent pouch can be worn comfortably by a user in a manner similar to wearing underwear. The pouch for collecting urine is disposable and is made of a soft comfortable absorbent material which prevents any moisture from leaking out. The pouch is provided with a vertical slit in a back wall facilitating the insertion of the user's penis into the interior of the pouch during use. The pouch is also provided with a flap which may be opened enabling the user to insert a hand into the pouch for guiding the penis therein. The pouch is closed during use sealing off the interior of the pouch, thereby preventing any leakage during sleeping.

### CROSS-REFERENCE TO RELATED APPLICATION

This application claims the benefit of Provisional Patent Application No. 61/246,552 filed on Sep. 29, 2009, in the United States Patent & Trademark Office, the disclosure of which is incorporated herein by reference.

### FIELD OF INVENTION

The present invention relates to absorbent pouches for managing male incontinence. More particularly, the present invention provides a disposable absorbent pouch for collecting urine of a male who is unable to get to a toilet.

### BACKGROUND OF THE INVENTION

Men may experience continence due to physical impairment caused by accidents or a plurality of medical conditions. Such males loose either partial or total control over urination leading to either a continual small flow of urine from the penis, or involuntary urination at a given time. Further, older males commonly experience one or both types of incontinence, due to ageing related medical conditions. Such males require assistance for management of the urinary continence. In a hospital environ the most commonly employed method is the use of external catheters which are connected to a disposable bag in which the user's urine is collected.

However, there are a host of problems associated with a prolonged use of external catheters. When using an external catheter, an individual with sensitive skin may encounter infections. Keeping the moisture away from the skin is vital in preventing sores and other forms of infection. External catheters may have a tendency to leak, which can cause irritation to the skin. The leak can also result in a time consuming cleanup. With a bed ridden or elderly patient it can be difficult to maneuver the individual in a way that will allow a catheter to be put on and taken off. Hence, a more efficient method of continence management is needed.

U.S. Pat. No. 6,132,412, issued on Oct. 17, 2000 to Jones; Rosemary N. describes a genitourinary elimination covering system which includes a shield member, which at its upper end includes a series of connecting means such as the hook-and-pile fastening means which connects the ends of the waist band tightly about the wearer, to sustain the shield in place, in order to form fit the pouch to the male anatomy, and assure its retention in place

Another U.S. Pat. No. 6,197,011, issued on Mar. 6, 2001 to Freitas, et al. describes a male incontinence diaper construction wherein the same can be utilized for absorbing urine from a male user. To attain this, the diaper generally comprises a back panel with a hole therethrough extending between the front and back faces of the back panel. A front flap is coupled to back panel along the side edges and the bottom edge of the back panel to cover the hole of the back panel. An elongate flexible belt strap is coupled to the back panel adjacent the top edge of the back panel.

Yet another U.S. Pat. No. 5,074,853, issued on Dec. 24, 2007 to Bryant; Tracy L. describes a diaper that is wrapped around the penis of a user only, so that adjacent body parts are not in contact with the inside portion of the diaper. The diaper permits a nurse to single-handedly slide it in place around the penis while the patient is either sitting or lying on his back without any need for lifting or moving him It is also shaped to provide a compact fit for comfort and tight adherence to the body to avoid the spreading of wetness during urination.

U.S. Pat. No. 7,192,424, issued on Mar. 20, 2007 to Cooper; Teresa describes a less invasive disposable urinary collection absorbent pouch for receiving a penis of a male patient comprises a freestanding bulb-shaped sack formed from a single sheet of laminate material. The sheet includes a first absorbent inner layer disposed between a moisture impervious outer layer and a second absorbent layer. The sack has a top hand insertion opening with elastic and an elastic passageway disposed orthogonal to the hand opening for securing the sack to the penis.

U.S. Pat. No. 6,132,412, issued on Oct. 17, 2000 to Jones; Rosemary N. describes a disposable type of covering or shield, which primarily is contoured and for use in conjunction with the male, and to provide a covering absorbent pouch for use for overlying the male anatomy, and to provide for elimination of urinary discharges, particularly amongst elderly patients, or the male who is incontinent.

Also, U.S. Pat. No. 5,695,485, issued on Dec. 9, 1997 to Duperret et al., describes a disposable, male incontinence absorbent pouch which allows for ease of applying and removing in a sitting, standing, or prone position either by the user himself, or by an attendant. This absorbent pouch, which accommodates both dribbling and total incontinence, includes a waterproof outer layer, a highly absorbent middle layer, and an inner layer of permeable, non-absorbent material to protect the wearer's penis from moisture.

Further, US Patent Application 20020177825, dated Nov. 28, 2002 to Scovel, Aldrene K. describes an incontinence absorbent pouch having a disposable urinary bag having a plurality of attachment means located on its outside surface for securement of the disposable urinary bag to the adjustable attachment belt. Housed within the disposable urinary bag is an absorbent material for urine absorption. The disposable urinary bag also has in its uppercentral region an elastic ring or seal that provides a barrier against urine when a penis is inserted into it.

Also, US Patent Application 20040106909, dated Jun. 3, 2004 to Browning, Robert L. describes an absorbent pouch for absorbing liquid due to mild or moderate male incontinence or post urinary drip. The absorbent pouch includes a waist strap, a pouch, a pocket and an insert for the pocket. The waist strap is secured around the waist of the user so that the pouch extends down along the front of the user and the pocket is adjacent the penis of the user.

Most of the continence management absorbent pouches and methods available are either cumbersome or uncomfortable to use, or are difficult for a user to use without any outside assistance. Hence, there is a need for a comfortable and effective replacement for an external catheter. There is need for an absorbent pouch which can easily be taken in to use for collecting urine for those that are unable to use a toilet such as those with limited mobility, bedridden patients, the elderly, and more. Also is needed an absorbent pouch which prevents leaks and accidents and effectively contains the urine, keeping moisture away from the skin. The present invention satisfies these needs, as well as others, and generally overcomes the deficiencies found in the background art.

### SUMMARY OF THE INVENTION

The present invention is directed towards male incontinence management absorbent pouches and provides an absorbent pouch for storing urine of a male suffering fro incontinence. A method of using the absorbent pouch is also provided.

In various embodiments of the present invention, an absorbent pouch for storing urine of males suffering from incontinence is provided. The absorbent pouch comprises: a first elastic band for fitting around a user's waist; a second elastic band having a first end and a second end, the first end of the second elastic band being attached to the first elastic band at a first position, the second end of the second elastic band being attached to the first elastic band at a second position, the first and second positions being adjustable; a pouch having a top open end and a bottom closed end, the top end being attachable to the first elastic band at a third position between the first and the second positions, the bottom end being attachable to the second elastic band, the pouch being made of an absorbent material.

In an embodiment, the pouch comprises: a flap covering the top end, lifting of the flap exposing an interior of the pouch; a front wall distal from the user's body; and a back wall proximate to the user's body, the back wall comprising a vertical slit for receiving the user's penis into the interior of the pouch during use. The absorbent pouch as claimed in claim 1 wherein the pouch is made of a soft comfortable fabric.

In an embodiment, the interior of the pouch comprises an absorbent material for absorbing the user's urine and preventing the urine from leaking out to exterior of the pouch. In another embodiment, the pouch is disposable after use. In another embodiment, the pouch is attachable to the first and second elastic bands using one or more fastening means.

A method for using an absorbent pouch for receiving urine of males suffering from incontinence is also provided. The absorbent pouch comprises: a first elastic band; a second elastic band having a first end and a second end being adjustably attachable to the first elastic band at a first position and a second position respectively; a pouch made of an absorbent material having a top open end and a bottom closed end. The method comprises the steps of: wearing the absorbent pouch by fitting the first elastic band around a waist of the user, inserting a right leg of the user in a space between the second elastic band and a right edge of the pouch and inserting a left leg of the user in a space between the second elastic band and a left edge of the pouch; adjusting the first and the second positions of attachment of the second elastic band to the first elastic band for securing the absorbent pouch snugly against the user's body while keeping the pouch in an upright position at a location facilitating insertion of the user's penis into the pouch; opening a flap covering the top open side of the pouch; inserting a hand into the pouch via the opened flap for guiding the user's penis into the pouch through an opening in a back wall of the pouch, the back wall being proximate to the user's body, the back wall comprising a vertical slit for receiving the user's penis into the interior of the pouch; closing the flap covering the top open side of the pouch for sealing off the pouch during use; removing the pouch after use for disposal; and reattaching a fresh pouch to the first and the second elastic bands prior to next use.

### BRIEF DESCRIPTION OF THE ACCOMPANYING DRAWINGS

The present invention is described by way of embodiments illustrated in the accompanying drawings wherein:

FIG. 1 illustrates the male incontinence absorbent pouch, in accordance with an embodiment of the present invention;

FIG. 2A illustrates a urine collecting pouch of the male incontinence absorbent pouch, in accordance with an embodiment of the present invention;

FIG. 2B illustrates the materials of a urine collecting pouch of the male incontinence absorbent pouch, in accordance with an embodiment of the present invention;

FIG. 3 illustrates a user inserting a hand into the urine collecting pouch of the male incontinence absorbent pouch, in accordance with an embodiment of the present invention;

FIG. 4 illustrates the male incontinence absorbent pouch being used by a user in a sitting posture, in accordance with another embodiment of the present invention;

FIG. 5 illustrates the male incontinence absorbent pouch being used by a user in a reclining posture, in accordance with an embodiment of the present invention;

FIG. 6A is a flowchart illustrating a method for using a male incontinence absorbent pouch without elastic bands, in accordance with an embodiment of the present invention; and

FIG. 6B is a flowchart illustrating a method for using a male incontinence absorbent pouch with elastic bands, in accordance with an embodiment of the present invention.

### DETAILED DESCRIPTION OF THE INVENTION

An absorbent pouch used for collecting urine for those males that are unable to get to a toilet is described herein. The present invention provides an absorbent pouch comprising a disposable pouch that can be worn by a male suffering from incontinence for urine collection. The absorbent pouch is easy to use, is non invasive and provides relief from uncomfortable and invasive urine disposable methods such as external catheters.

The following disclosure is provided in order to enable a person having ordinary skill in the art to practice the invention. Exemplary embodiments are provided only for illustrative purposes and various modifications will be readily apparent to persons skilled in the art. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

The present invention would now be discussed in context of embodiments as illustrated in the accompanying drawings.

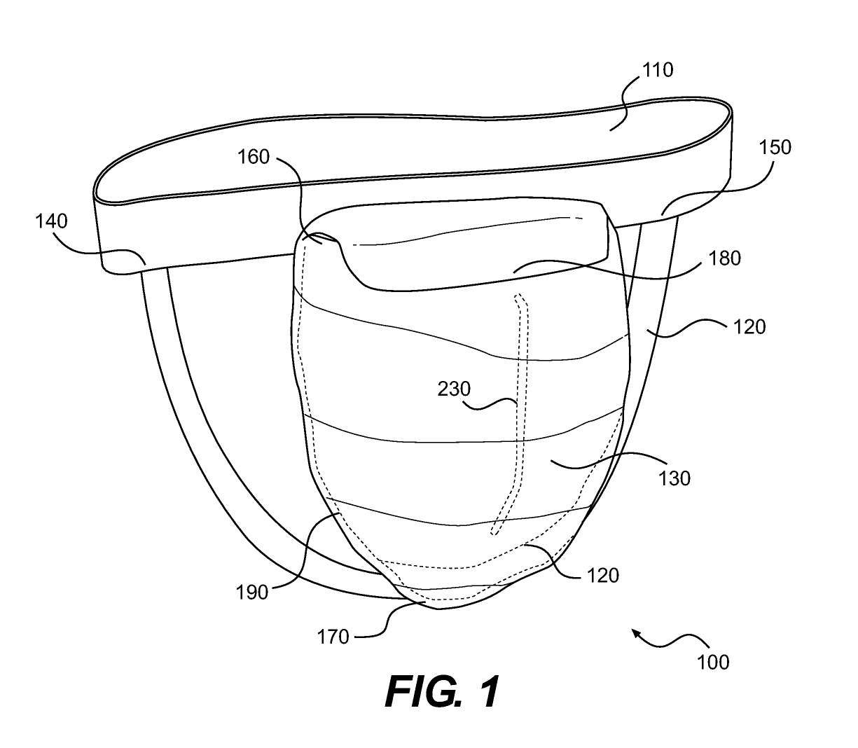

FIG. 1 illustrates the male incontinence absorbent pouch, in accordance with an embodiment of the present invention. The absorbent pouch 100 comprises a circular elastic band 110, a second elastic band 120 and a pouch 130. The circular elastic band 110 and the second elastic band 120 are made from any comfortable elastic material known in the art. The circular elastic band 110 is designed to fit around the waist of a user. In various embodiments, the absorbent pouch 100 maybe available in varying sizes of the elastic bands 110 and 120 to fit users of varying weights and waist sizes. The elastic bands are made of natural, butyl, or synthetic rubber.

The second elastic band 120 is attached to the elastic band 110 at a first position 140 and at a second position 150. In an embodiment, the positions of attachment 140 and 150 are adjustable around the length of the circular elastic band 110, enabling a user to wear the absorbent pouch 100 in a manner similar to wearing an underwear, such that the pouch 130 fits snugly against his body in a position facilitating the insertion of the user's penis into the pouch 130. The user may put on the absorbent pouch 100 by inserting his right and left legs in the space between the elastic band 120 and the right and left edges of the pouch 130 respectively, and then adjusting the positions of attachment 140 and 150 for a snug fit. In another embodiment the positions of attachment 140 and 150 are fixed and cannot be adjusted.

In an embodiment, the exterior of the pouch 130 is made of a soft fabric material, while the interior is made of any absorbent material known in the art for absorbing the user's urine and preventing any leakage of moisture to the exterior of the pouch 130.

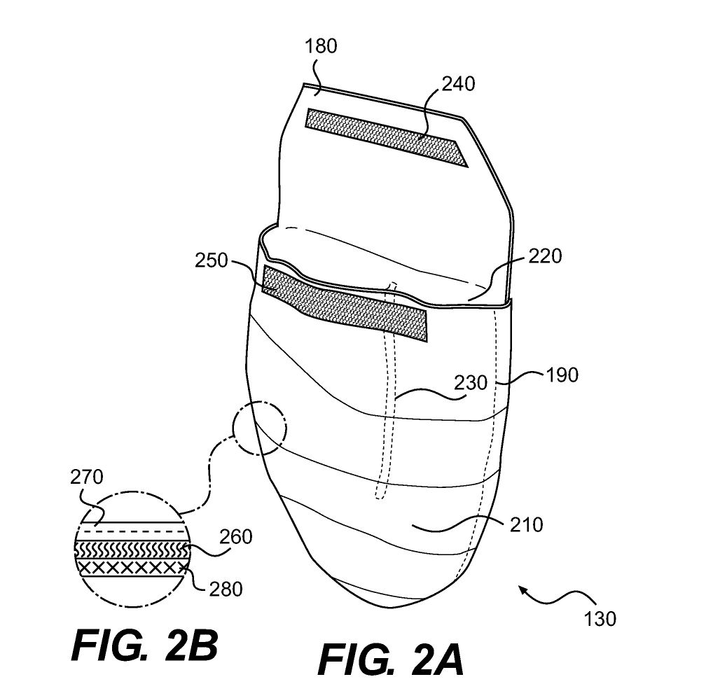

The pouch 130 comprises an open top end 160 and a closed bottom end 170. The open end 160 is attached to the circular elastic band 110 at a location in between the positions of attachment 140 and 150 of the second elastic band 120. The bottom end 170 is attached to the second elastic band 120 at a location enabling the pouch to remain in an upright position as illustrated in FIG. 1. In various embodiments, the pouch 130 is attached to the elastic bands 110 and 120 by using fastening means such as hooks, fasteners, Velcro, etc. that allow the user to easily attach the pouch before use and just as easily remove the pouch for disposal after use. The top end 160 of the pouch 130 is provided with a flap 180 which may be opened for accessing the interior of the pouch 130 and closed at other times. The flap 180 is described in conjunction with FIG. 2A.

FIG. 2A illustrates a urine collecting pouch of the male incontinence absorbent pouch, in accordance with an embodiment of the present invention. The pouch 130 comprises a front wall 210 and a back wall 220 (visible in FIG. 2A). A vertical slit 230 (visible in FIGS. 1-3) is provided in the back wall 220. The vertical slit 230 is about 2-3 inches in length. The back wall 220 is proximate to the body of the user wearing the absorbent pouch 100 while the front wall 210 is distal to his body. The vertical slit 230 facilitates insertion of the user's penis into the interior of the pouch 130 for urination. FIG. 2A also illustrates the flap 180 which is used for closing the interiors of the pouch 130 by using any suitable fastening means such as hooks, fasteners, Velcro, etc. that allow the user to easily open the pouch for inserting a hand for guiding his penis through the vertical slit 230 and just as easily close the flap 180 before urination. A sewn line 190 (e.g. adhesive) is provided for edge attachment of the front wall 210 and the back wall 220 of the pouch 100.

As in FIG. 2B, the material of the interior region 260 of the pouch 130 absorbs moisture while still keeping an outer layer 270 and a breathable back sheet 280 of the pouch which is in contact with the user's skin, dry. The outer layer 270 is made from a moisture impermeable or liquid resistant material (e.g. polypropylene film). The inner layer 260 is formed from liquid absorbent material, fluff material, cellulose wadding, absorbent cotton fibers, polyester or polyolefin mixed combination. Multi-layers of absorbent materials can also be used together. Back sheet 280 can be made from highly porous nonwoven fabrics or paper-based porous materials.

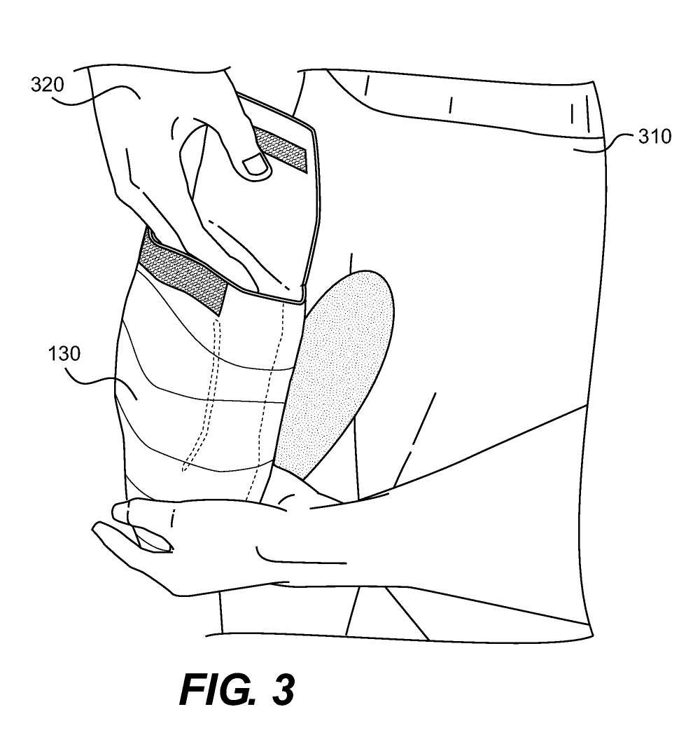

FIG. 3 illustrates a user inserting a hand into the urine collecting pouch 130 of the male incontinence absorbent pouch 100, in accordance with an embodiment of the present invention. In various embodiments, the user 310 may use his hand 320 for inserting or removing his penis from the pouch 130 through the vertical slit in the back wall of the pouch 130, or for resting his penis in a more comfortable position within the interior of the pouch. With reference to FIG. 2 the flap 180 is provided with a Velcro strip 240 which is stuck onto another Velcro strip 250 provided on the front wall 220 of the pouch 130, in order to close the top end of the pouch 130. In other embodiments of the present invention various other means known in the art for fastening the flap 180 onto the front wall 220 of the pouch 130 may be used.

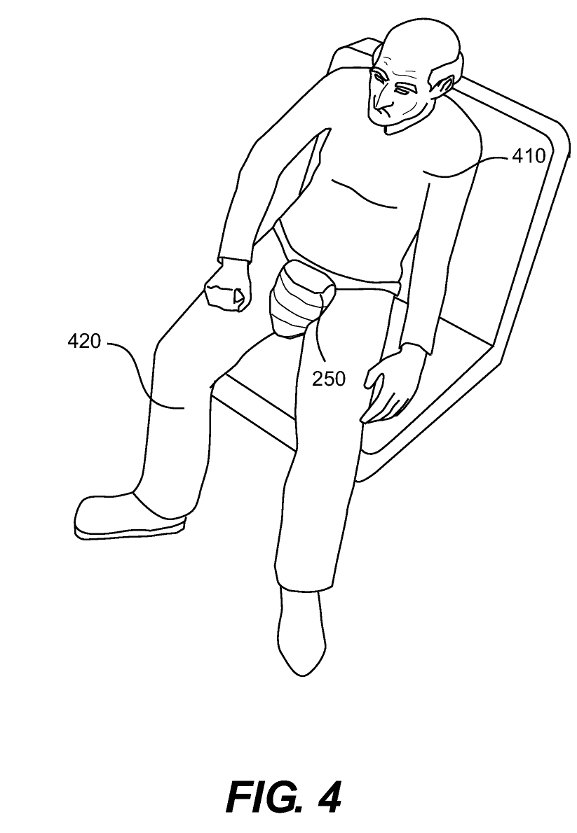

FIG. 4 illustrates the male incontinence absorbent pouch being used by a user in a sitting posture, in accordance with another embodiment of the present invention. As illustrated in FIG. 4, in an embodiment, the absorbent pouch 100 can be worn by a user 410 over another piece of garment such as pants 420. When wearing the absorbent pouch 100 over another garment, the user is required to guide the penis through an opening that is often present on male clothing, sometimes referred to as the fly. In other embodiments, the absorbent pouch 100 may be worn under clothes on a naked body of a user.

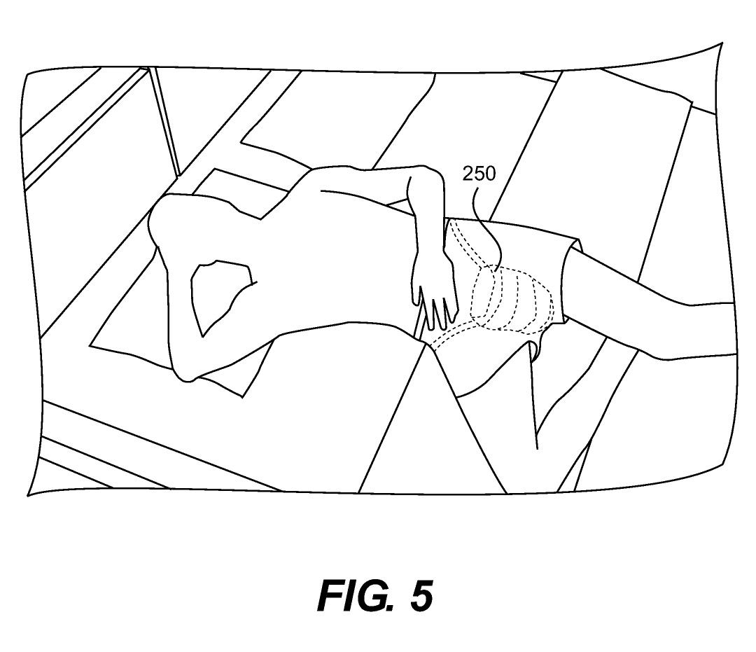

FIG. 5 illustrates the male incontinence absorbent pouch being used by a user in a reclining posture, in accordance with an embodiment of the present invention. As illustrated in FIG. 5, the absorbent pouch 100 is comfortable to wear and enables a user to relax in any posture while still wearing the absorbent pouch 100. With reference to FIG. 1, in various embodiments, after every mination or after a predefined period of time, the pouch 130 may be removed from the elastic bands 110 and 120, and disposed off. A fresh pouch 130 may then be attached to the bands 110 and 120, either while the user is still wearing the bands 110, 120, or by taking off the bands 110 and 120, attaching a fresh pouch 130 and wearing the absorbent pouch 100 again. In another embodiment, the entire absorbent pouch 100 is required to be disposed after use and a fresh absorbent pouch donned on before next use. The pouch 100 can be changed or removed without removing the wearer's diaper or boxer.

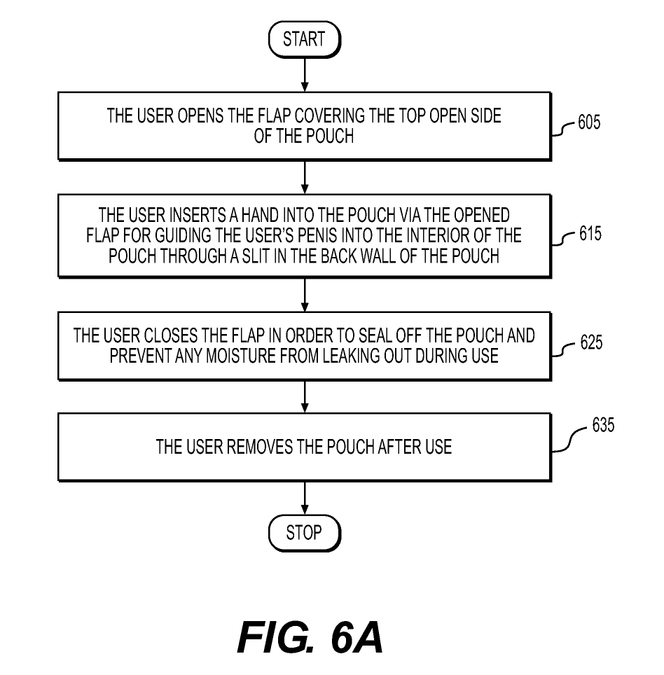

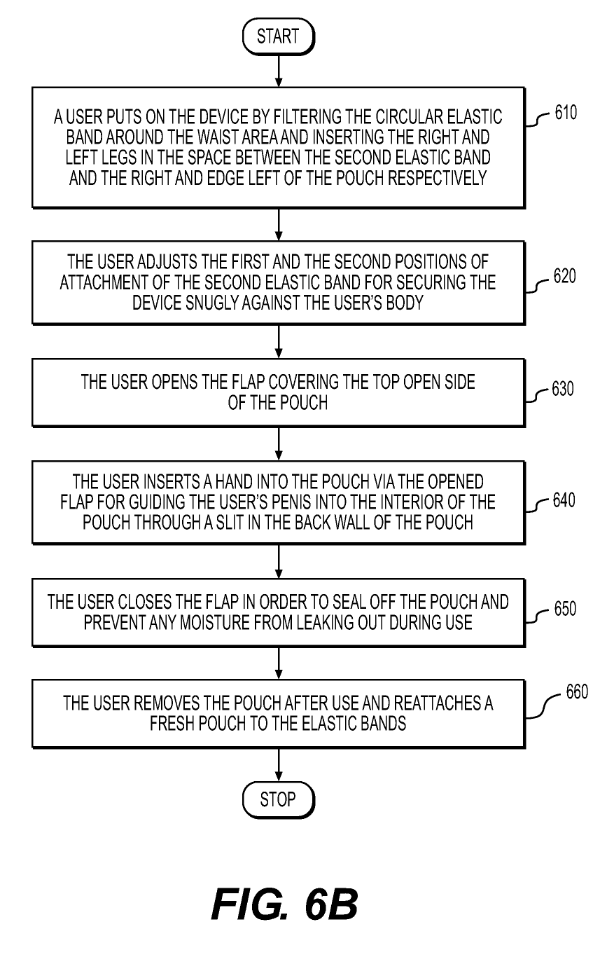

FIG. 6A is a flowchart illustrating a method for using a male incontinence absorbent pouch without using elastic bands, in accordance with an embodiment of the present invention. FIG. 6A illustrates a method of using the absorbent pouch 100 described in conjunction with FIGS. 1-5. In order to commence using the absorbent pouch 100, at step 605, the user opens the flap 180 covering the top open side 160 of the pouch. At step 615, the user inserts a hand into the pouch 130 via the opened flap 180 for guiding the user's penis into the interior of the pouch 130 through a vertical slit 230 in the back wall 220 of the pouch. At step 625, the user closes the flap 180 in order to seal off the pouch 130 and prevent any moisture from leaking out during use. At step 635, the user removes the pouch 130 after use and reattaches a fresh pouch to the elastic bands 110 and 120. In an embodiment, the user disposes off the entire absorbent pouch 100 after use and puts on a fresh absorbent pouch before next use.

FIG. 6B is a flowchart illustrating a method for using a male incontinence absorbent pouch with elastic bands, in accordance with an embodiment of the present invention. FIG. 6B illustrates a method of using the absorbent pouch 100 described in conjunction with FIGS. 1-5. In order to commence using the absorbent pouch 100, at step 610, a user puts on the absorbent pouch 100 in a manner similar to wearing an underwear by fitting the circular elastic band 110 around the waist area and inserting the right and left legs in the space between the second elastic band 120 and the right and left edge of the pouch 130 respectively. At step 620 the user adjusts the first and the second positions 140, 150 of attachment of the second elastic band 120 for securing the absorbent pouch snugly against the user's body while keeping the pouch 130 in an upright position at a location facilitating insertion of the user's penis into the pouch. At step 630, the user opens the flap 180 covering the top open side 160 of the pouch. At step 640, the user inserts a hand into the pouch 130 via the opened flap 180 for guiding the user's penis into the interior of the pouch 130 through a vertical slit 230 in the back wall 220 of the pouch. At step 650, the user closes the flap 180 in order to seal off the pouch 130 and prevent any moisture from leaking out during use. At step 660, the user removes the pouch 130 after use and reattaches a fresh pouch to the elastic bands 110 and 120. In an embodiment, the user disposes off the entire absorbent pouch 100 after use and puts on a fresh absorbent pouch before next use.

Therefore the present invention provides a comfortable and effective replacement for an external catheter. The absorbent pouch of the present invention is designed to collect urine for those males that are unable to use a toilet such as those with limited mobility, bedridden patients, the elderly, and more. The male incontinence absorbent pouch can be put on quickly, disposed of easily and also prevents leaks and accidents. The pouch of the present absorbent pouch effectively contains the urine, thereby keeping moisture away from the skin of the user. The pouch can be changed or removed without removing the wearer's diaper or boxer.

While the exemplary embodiments of the present invention are described and illustrated herein, it will be appreciated that they are merely illustrative. It will be understood by those skilled in the art that various changes in form and detail may be made therein without departing from or offending the spirit and scope of the invention as defined by the appended claims.

What is claimed is:

1. A absorbent pouch system for storing urine of males suffering from incontinence, the absorbent pouch system comprising:
a. a first elastic band for fitting around a user's waist;
b. a second elastic band having a first end and a second end, the first end of the second elastic band being attached to the first elastic band at a first position, the second end of the second elastic band being attached to the first elastic band at a second position, the first and second positions being adjustable;
c. a pouch having a top open end and a bottom closed end, the top end being attachable to the first elastic band at a third position between the first and the second positions, the bottom end being attachable to the second elastic band, the pouch being made of an absorbent material, the pouch comprising:
i. a flap covering the top end, lifting of the flap exposing an interior of the pouch;
ii. a front wall distal from the user's body;
iii. a back wall proximate to the user's body, the back wall comprising a vertical slit for receiving the user's penis into the interior of the pouch during use; and
iv. a sewn line is provided for edge attachment between the front wall and the back wall.

2. The absorbent pouch as claimed in claim 1 wherein the pouch is made of three layers: liquid permeable outer layer, an absorbent inner layer and a breathable back sheet.

3. The absorbent pouch as claimed in claim 2, wherein the outer layer is made from a moisture impermeable or liquid resistant material such as polypropylene film.

4. The absorbent pouch as claimed in claim 2, wherein the inner layer is formed from liquid absorbent material, fluff material, cellulose wadding, absorbent cotton fibers, polyester or polyolefin mixed combination.

5. The absorbent pouch as claimed in claim 4, wherein multi-layers of absorbent materials can also be used together in the inner layer.

6. The absorbent pouch as claimed in claim 2, wherein the breathable back sheet can be made from highly porous nonwoven fabrics or paper-based porous materials.

7. The absorbent pouch as claimed in claim 1, wherein the elastic bands are optional.

8. The absorbent pouch as claimed in claim 1, wherein the vertical slit is 2-3 inches in length.

9. The absorbent pouch as claimed in claim 1 wherein the interior of the pouch comprises an absorbent material for absorbing the user's urine and preventing the urine from leaking out to exterior of the pouch.

10. The absorbent pouch as claimed in claim 1 wherein the pouch is disposable after use.

11. The absorbent pouch as claimed in claim 1 wherein the pouch is attachable to the first and second elastic bands using one or more fastening means.

12. A method for using a absorbent pouch for receiving urine of males suffering from incontinence, the absorbent pouch comprising: a first optional elastic band; a second optional elastic band having a first end and a second end being adjustably attachable to the first elastic band at a first position and a second position respectively; a pouch made of an absorbent material having a top open end and a bottom closed end, the method comprising the steps of:
a. wearing the absorbent pouch by fitting the first optional elastic band around a waist of the user, inserting a right leg of the user in a space between the second optional elastic band and a right edge of the pouch and inserting a left leg of the user in a space between the second elastic band and a left edge of the pouch;
b. adjusting the first and the second positions of attachment of the second elastic band to the first elastic band for securing the absorbent pouch snugly against the user's body while keeping the pouch in an upright position at a location facilitating insertion of the user's penis into the pouch;
c. opening a flap covering the top open side of the pouch;
d. inserting a hand into the pouch via the opened flap for guiding the user's penis into the pouch through an opening in a back wall of the pouch, the back wall being proximate to the user's body, the back wall comprising a vertical slit for receiving the user's penis into the interior of the pouch;
e. closing the flap covering the top open side of the pouch for sealing off the pouch during use;
f. removing the pouch after use for disposal; and
g. re-attaching a fresh pouch to the first and the second elastic bands prior to next use.
